(19) United States
(12) Patent Application Publication
(10) Pub. No.: US 2008/0269617 A1
(43) Pub. Date: Oct. 30, 2008

(54) Absorption and Scattering Map Reconstruction For Optical Fluorescence Tomography

(75) Inventors: Thomas Kohler, Norderstedt (DE); Tim Nielsen, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/092,881

(22) PCT Filed: Oct. 24, 2006
(86) PCT No.: PCT/IB06/53908

§ 371 Date: May 7, 2008

(30) Foreign Application Priority Data
Nov. 10, 2005 (EP) 05110585.6

(51) Int. Cl. A61B 5/00

(52) U.S. Cl. 600/476

(57) ABSTRACT

Optical fluorescence tomography is a highly sensitive method to image contrast agents in the body. However, current reconstruction methods suffer from a high complexity or even instability. According to an exemplary embodiment of the present invention, a method for absorption/scattering map reconstruction for optical fluorescence tomography may be provided, which uses a spectral model. This may provide for a subsequent fluorescence reconstruction with improved image quality.

The present invention relates to the field of fluorescence tomography. In particular, the present invention relates to an optical fluorescence tomography apparatus for examining an object of interest, an image processing device, a computer-readable medium, a program element and a method of examination of an object of interest.

Optical fluorescence tomography is a highly sensitive method for imaging contrast agents in the body. However, the distribution of contrast agent may only be possible if, during the reconstruction process, the absorption and scatter coefficients of the surrounding tissue are known at the fluorescence wavelength.

In optical fluorescence tomography, the light propagation through the tissue is typically described by the diffusion equation

Where λₓ is the wavelength of the excitation light, λ_{f} the wavelength of the fluorescence light, μₐ is the absorption coefficient of the tissue, μ_{c} is the absorption coefficient of the contrast agent. Φₓ and Φ_{f} are the intensities of the excitation and emission light, respectively. Finally, D=1/(3(1−g)μₛ) is the diffusion coefficient (μₛ is the scatter coefficient) and q₀ is the source term, i.e. the term that models the injection of excitation light. Fluorescence light is generated by the contrast agent with efficiency γ, which is a linear function of the concentration of the contrast agent. All quantities except the wavelength are spatially variant.

The task of optical fluorescence tomography is to reconstruct the spatial distribution of the contrast agent γ based on measurement of Φₓ and Φ_{f} at the surface of the object.

The above set of two coupled partial differential equations (PDE) can be decoupled by performing first transmission measurements (described by the first equation only) in order to determine some of the parameters needed in the second equation, viz. D(λ_{f}), μₐ(λ_{f}) and Φₓ. These values are used in the second equation to reconstruct γ from measurements of the fluorescence light Φ_{f}.

In order to provide for a knowledge of the absorption and scatter coefficients of the surrounding material or tissue, a simultaneous reconstruction of both, the emission and absorption/scattering maps may be performed. Alternatively, a beforehand reconstruction of the absorption/scattering map may be performed on the basis of a single transmission measurement which is taken at the excitation wavelength.

However, simultaneous reconstruction may suffer from a high complexity. Furthermore, both methods may suffer from a high instability, which may result in a reconstruction of the maps with low spatial resolution.

It may be desirable to have an improved absorption/scattering map reconstruction.

According to an exemplary embodiment of the present invention, an optical fluorescence tomography apparatus for examining an object of interest may be provided, the optical fluorescence tomography apparatus comprising a detector unit adapted for detecting first light transmitted through the object of interest and second light emitted by a contrast agent inside the object of interest, resulting in detection data, and a reconstruction unit adapted for performing a fluorescence reconstruction on the basis of the detection data and a spectral model, resulting in reconstruction data comprising the spatial distribution of the contrast agent inside the object of interest.

Thus, according to this exemplary embodiment of the present invention, a spectral model may be used for fluorescence reconstruction of detection data. This may improve the stability of the reconstruction of the optical properties of the object of interest (which may, for example, be tissue).

According to another exemplary embodiment of the present invention, the spectral model comprises a first concentration of a contrast agent.

Furthermore, according to another exemplary embodiment of the present invention, the spectral model comprises a second concentration of oxygenated haemoglobin, a third concentration of deoxygenated haemoglobin, and a fourth concentration of water.

Thus, by including the concentration of the contrast agent into the model, the absorption and scattering at the fluorescence wavelength may be derived with higher stability.

According to another exemplary embodiment of the present invention, the spectral model comprises an absorption model and a scattering model, wherein the reconstruction unit is further adapted for determining an absorption at a fluorescence wavelength on the basis of the absorption model and for determining a scattering at the fluorescence wavelength on the basis of the scattering model.

Therefore, according to this exemplary embodiment of the present invention, both, an absorption map at the fluorescence wavelength and a scattering map at the fluorescence wavelength may be determined on the basis of the spectral model.

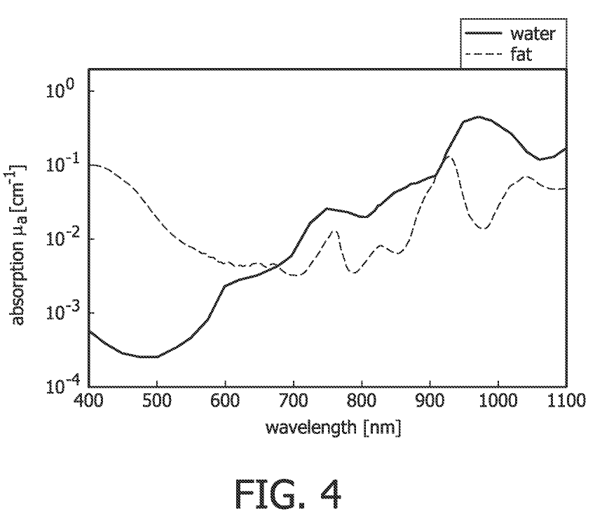

According to another exemplary embodiment of the present invention, the absorption model reads

in which λ is a wavelength, N is a number of chromophores in the model, Cᵢ is a concentration of the chromophore i, and ε(i, λ) is an absorption of chromophore i at wavelength λ. The spectral dependence of the absorption of typical constituents of tissue are known and shown in the graph for water and fat, which is depicted in FIG. 4.

This may provide for a direct reconstruction of the concentration of chromophores using near-infrared transmission measurements.

Furthermore, according to another exemplary embodiment of the present invention, the scattering model reads

wherein A is a scatter amplitude and B is a scatter power.

Thus, by using this global model, a determination of a scattering map may be provided.

According to another exemplary embodiment of the present invention, the optical fluorescence tomography apparatus comprises an excitation source adapted for emitting electromagnetic radiation to the object of interest. For example, the radiation emitted by the excitation source may be near-infrared light. Furthermore, the excitation source may be adapted for emitting time-varying, i.e. modulated, excitation light to the object of interest. Then, lock-in techniques may be used. Furthermore, the amplitude and the phase shift of the transmitted light may be detected independently in order to obtain additional information about the scattering and absorption of the object.

Furthermore, filters may be provided in order to filter emission light from the object of interest in order to reject transmitted excitation light.

According to another exemplary embodiment of the present invention, the excitation source and the detector unit are adapted for moving around the object of interest. This may provide for tomographic three-dimensional detection data acquired from different source and detector positions.

According to another exemplary embodiment of the present invention, the optical fluorescence tomography apparatus is configured as one of the group consisting of a medical application apparatus or a material testing apparatus.

According to another exemplary embodiment of the present invention, an image processing device for examination of an object of interest may be provided, the image processing device comprising a memory for storing detection data of the object of interest. Furthermore, the image processing device may comprise a reconstruction unit adapted for performing a fluorescence reconstruction on the basis of the detection data and a spectral model, resulting in reconstruction data comprising optical properties of the object of interest.

Therefore, an image processing device may be provided which is adapted for performing an improved fluorescence reconstruction on the basis of an absorption and scattering map reconstruction.

According to another exemplary embodiment of the present invention, a method of examination of an object of interest with an optical fluorescence tomography apparatus may be provided, the method comprising the steps of emitting, by an excitation source, electromagnetic radiation to the object of interest, detecting, by a detector unit, first light transmitted through the object of interest and second light emitted by a contrast agent inside the object of interest, resulting in detection data, and performing, by a reconstruction unit, a fluorescence reconstruction on the basis of the detection data and a spectral model, resulting in reconstruction data comprising a spatial distribution of the contrast agent inside the object of interest.

According to another exemplary embodiment of the present invention, a computer-readable medium may be provided, in which a computer program of examination of an object of interest is stored which, when being executed by a processor, is adapted to carry out the above-mentioned method steps.

Furthermore, the present invention relates to a program element of examination of an object of interest, which may be stored on the computer-readable medium. The program element may be adapted to carry out the steps of emitting electromagnetic radiation, e.g. near infra-red light, to the object of interest, detecting transmitted light through the object of interest and/or light emitted by a contrast agent inside the object of interest and performing a fluorescence reconstruction on the basis of the detection data and the spectral model.

The program element may be preferably be loaded into working memories of a data processor. The data processor may thus be equipped to carry out exemplary embodiments of the methods of the present invention. The computer program may be written in any suitable programming language, such as, for example, C++ and may be stored on a computer-readable medium, such as a CD-ROM. Also, the computer program may be available from a network, such as the WorldWideWeb, from which it may be downloaded into image processing units or processors, or any suitable computers.

It may be seen as the gist of an exemplary embodiment of the present invention that a reconstruction of absorption and scattering maps at the fluorescence wavelength for optical fluorescence tomography is performed by using a spectral model. According to an aspect of the present invention this map is then used for a subsequent fluorescence reconstruction which may result in an improved image quality.

These and other aspects of the present invention will become apparent from and elucidated with reference to the embodiments described hereinafter.

Exemplary embodiments of the present invention will be described in the following, with reference to the following drawings.

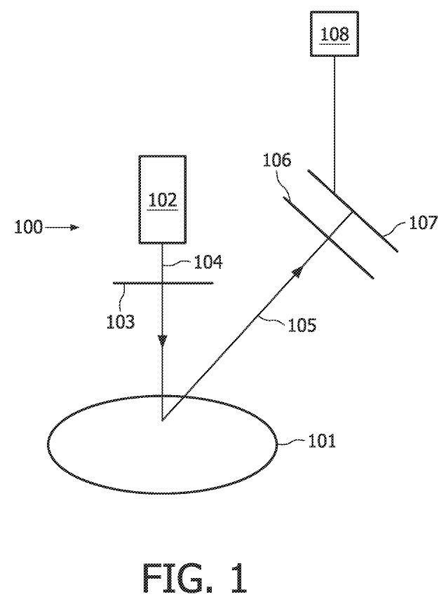

FIG. 1 shows a simplified schematic representation of an optical fluorescence tomography apparatus according to an exemplary embodiment of the present invention.

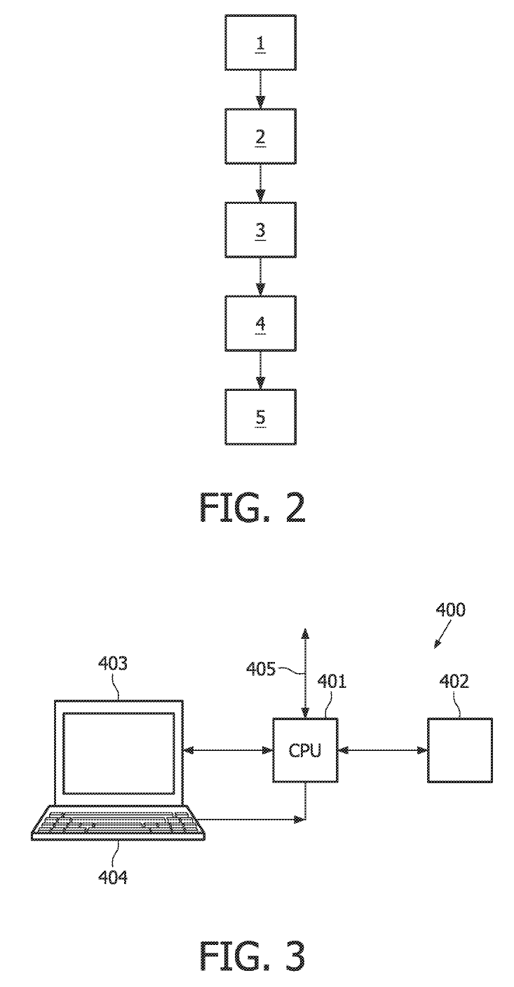

FIG. 2 shows a flow-chart of an exemplary embodiment of a method according to the present invention.

FIG. 3 shows an exemplary embodiment of an image processing device according to the present invention, for executing an exemplary embodiment of a method in accordance with the present invention.

FIG. 4 shows the graph for water and fat, from which the spectral dependence of the absorption of typical constituents of tissue can be derived.

The illustration in the drawings is schematically. In different drawings, similar or identical elements are provided with the same reference numerals.

FIG. 1 shows a simplified schematic representation of an embodiment of a fluorescence tomography apparatus for optical examination of an object of interest. The fluorescence tomography apparatus 100 generates three-dimensional images of the object of interest 101 (for example tissue) based on detection data and a spectral model.

Visible light and near-infrared light interact with biological tissue predominantly by absorption and elastic scattering. An examination system according to an exemplary embodiment of the present invention quantifies intrinsic tissue chromophore concentrations and scattering properties, thereby providing valuable functional information. Such an examination system measures light transmission, e.g. in the near-infrared, and utilizes model-based computational methods in order to generate spatially-resolved absolute images of oxyhemoglobin, deoxyhemoglobin, and water, as well as scattering parameters affected by cellular and sub-cellular structural elements.

The excitation source 102 is adapted for emitting excitation light 104 to the object of interest 101, resulting in an excitation of fluorescence targets inside the object of interest 101. The excitation source 102 may be adapted in form of a laser diode, which may be adapted for generating intensity-modulated or pulsed excitation light or constant excitation light. For example, the laser diode may be adapted for emitting near-infrared excitation light having a wavelength of, for example, 700 nm to 900 nm. However, light source 102 may be adapted for emitting light of other wavelengths. The same light source is used to emit light at further wavelengths, for example eight different wavelengths between 600 nm and 900 nm, in order to perform the measurements required for the reconstruction of the chromophores. This may be implemented by using different laser diodes and a fiber switch to couple the light of the different laser diodes into the object. In yet another exemplary embodiment, laser diodes with different wavelengths may be used as independent light sources, meaning that they inject the light at different locations.

The apparatus may comprise a lens-, fiber, or filter-system 103 adapted for generating an expanded beam of excitation light for illuminating the object of interest 101.

The object of interest 101 may comprise a fluorescent contrast agent adapted for emitting light in response to the excitation light 104. The detected light 105 comprises emission light at the fluorescence wavelength and/or transmitted light at the incident wavelength 104. For example in order to minimize the excitation light beam pre-processing unit 106 may be used. The pre-processing unit 106 may comprise filter elements, such as band pass filters, band rejection filters, both for example coupled via a lens, multiple lenses, and/or a collimator.

After passing the beam pre-processing unit 106, the light 105 from the object of interest 101 hits the detector 107, which, for example, may be adapted in form of an intensified charge coupled camera (CCD) or a photodiode. Furthermore, the detector 107 may be coupled to a reconstruction unit 108 adapted for performing the fluorescence reconstruction in order to provide for a three-dimensional image.

FIG. 2 shows an exemplary embodiment of a method according to the present invention for performing an absorption/scattering map reconstruction for optical fluorescence tomography, which may result in an improved image.

The method starts with step 1 in which the excitation source emits electromagnetic radiation of, e.g., eight different wavelengths to the object of interest. Thus, the source may comprise eight different sub-sources, e.g. laser diodes, each emitting a different wavelength of infrared light. The eight radiation beams may then be coupled by a coupler, e.g. a fibre coupler or a system of lenses before the coupled light is directed to the object of interest.

Then, in step 2, the emitted light reaches the object of interest and is transmitted. The transmitted light is detected by the detector unit and the parameters Cᵢ, A and B are determined on the basis of the detected transmission data.

From these parameters, in step 3, absorption/absorption and scattering at the fluorescent wavelength (i.e. the optical properties of the object of interest at the fluorescent wavelength) are calculated on the basis of an absorption model and the scattering model, respectively.

Furthermore, in step 4, the contrast agent inside the object of interest is excited and the light emitted by the contrast agent is detected by the detector unit. The calculated values for absorption and scattering at this wavelength are used for reconstructing the spatial distribution of the contrast agent inside the object of interest.

It should be noted, that step 4 may be performed consecutively or parallel to steps 2 and 3 by using spectrally resolved detection systems.

By using a spectral model for the absorption and the scattering in order to reconstruct directly the concentration of chromophores using near-infrared transmission measurements at several distinct frequencies, the stability of the reconstruction may be significantly improved compared with the separate reconstruction of the transmission data from each individual frequency. The spectral model for the absorption reads

wherein λ is the wavelength, N is the number of chromophores in the model, Cᵢ is the concentration of the chromophore i, and ε(i, λ) is the absorption of chromophore i at wavelength λ. Furthermore, scattering may be modelled with the global model

wherein A is the scatter amplitude and B is the scatter power. This model may not only be used for transmission tomography measurements to reconstruct the concentration of the constituents, but also, according to an aspect of the present invention, in fluorescence tomography in order to improve the stability of the reconstruction of the spatial distribution of the contrast agent.

For example, the spectral model (which may contain the concentration of oxygenated and deoxygenated haemoglobin and water) may be extended by the concentration of the contrast agent. From the model, the absorption and scattering at the fluorescence wavelength may be derived with higher stability. This implies that it may be reconstructed with less artefacts and the subsequent fluorescence reconstruction may be performed with improved accuracy (in step 5).

It should be noted that the concentration of the chromophores may, according to an exemplary embodiment of the present invention, only be used as an intermediate quantity.

FIG. 3 shows an exemplary embodiment of an image processing device according to the present invention for executing an exemplary embodiment of the method in accordance with the present invention. The image processing device 400 depicted in FIG. 3 comprises a central processing unit (CPU) or image processor 401 connected to a memory 402 for storing an image depicting an object of interest, such as a patient or a material to be analyzed. The data processor 401 may be connected to a plurality of input/output network or diagnosis devices, such as an optical fluorescence tomography apparatus. The data processor 401 may be furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 3.

FIG. 4 shows the graph for water and fat, from which the spectral dependence of the absorption of typical constituents of tissue can be derived.

The examination of an object of interest according to the present invention may allow for an improvement of the stability of the reconstruction of the optical properties of the tissue, which may result in an improvement of the subsequent fluorescence reconstruction.

Exemplary embodiments of the invention may be sold as a software option for an optical mammography scanner console, imaging work stations or PACS work stations.

It should be noted that the term “comprising” does not exclude other elements or steps and the “a” or “an” does not exclude a plurality and that a single processor or system may fulfill the functions of several means or units recited in the claims. Also elements described in association with different embodiments may be combined.

It should also be noted, that any reference signs in the claims shall not be construed as limiting the scope of claims.

1. An optical fluorescence tomography apparatus (100) for examining an object of interest (101), the optical fluorescence tomography apparatus (100) comprising:
a detector unit (107) adapted for detecting first light transmitted through the object of interest (101) and second light emitted by a contrast agent inside the object of interest (101), resulting in detection data; and
a reconstruction unit (108) adapted for performing a fluorescence reconstruction on the basis of the detection data and a spectral model, resulting in reconstruction data comprising a spatial distribution of the contrast agent inside the object of interest (101).

2. The optical fluorescence tomography apparatus of claim 1, wherein the spectral model comprises a first concentration of a contrast agent.

3. The optical fluorescence tomography apparatus of claim 1, wherein the spectral model comprises an absorption model and a scattering model; wherein the reconstruction unit (108) is further adapted for:
determining a spatial distribution of absorption at a fluorescence wavelength on the basis of the absorption model; and
determining a spatial distribution of scattering at the fluorescence wavelength on the basis of the scattering model.

4. The optical fluorescence tomography apparatus of claim 3, wherein the absorption model reads
$${μ}_{a} ( λ , \vec{x} ) = {\sum }_{i = 1}^{N} {C}_{i} ( \vec{x} ) ɛ ( i , λ )$$
wherein λ is a wavelength, N is a number of chromophores in the model, Cᵢ is a concentration of the chromophore i, and ε(i, λ) is an absorption of chromophore i at wavelength λ.

5. The optical fluorescence tomography apparatus of claim 3, wherein the scattering model reads
μ′ₛ(λ, \vec{x})=A(\vec{x})λ^{B(\vec{x})}
wherein A is a scatter amplitude and B is a scatter power.

6. The optical fluorescence tomography apparatus of claim 2, wherein the spectral model comprises a second concentration of oxygenated haemoglobin, a third concentration of deoxygenated haemoglobin, and a fourth concentration of water.

7. The optical fluorescence tomography apparatus of claim 1, wherein the detector unit (107) comprises detecting elements; wherein each detecting element is adapted for detecting the transmitted or emitted light in an spectrally resolved manner.

8. The optical fluorescence tomography apparatus of claim 1, further comprising an excitation source (102) adapted for emitting electromagnetic radiation to the object of interest (101).

9. The optical fluorescence tomography apparatus of claim 1, wherein the excitation source (102) and the detector unit (107) are adapted for moving around the object of interest (101).

10. An image processing device for examination of an object of interest (101), the image processing device comprising:
a memory for storing detection data of the object of interest (101), the detection data comprising detected first light transmitted through the object of interest (101) and detected second light emitted by a contrast agent inside the object of interest (101); and
a reconstruction unit (108) adapted for performing a fluoresce reconstruction on the basis of the detection data and a spectral model, resulting in reconstruction data comprising a spatial distribution of the contrast agent inside the of the object of interest (101).

11. A computer-readable medium (402), in which a computer program of examination of an object of interest (101) is stored which, when being executed by a processor (401), is adapted to carry out the steps of:
emitting, by an excitation source (102), electromagnetic radiation to the object of interest (101);
detecting, by a detector unit (107), first light transmitted through the object of interest (101) or second light emitted by a contrast agent inside the object of interest (101), resulting in detection data; and
performing, by a reconstruction unit (108), a fluoresce reconstruction on the basis of the detection data and a spectral model, resulting in reconstruction data comprising a distribution of the contrast agent inside the object of interest (101).

12. A program element of examination of an object of interest (101), which, when being executed by a processor (401), is adapted to carry out the steps of:
emitting, by an excitation source (102), electromagnetic radiation to the object of interest (101);
detecting, by a detector unit (107), first light transmitted through the object of interest (101) or second light emitted by a contrast agent inside the object of interest (101), resulting in detection data; and
performing, by a reconstruction unit (108), a fluoresce reconstruction on the basis of the detection data and a spectral model, resulting in reconstruction data comprising a distribution of the contrast agent inside the object of interest (101).

13. Method of examination of an object of interest with an optical fluorescence tomography apparatus (100), the method comprising the steps of:
emitting, by an excitation source (102), electromagnetic radiation to the object of interest (101);
detecting, by a detector unit (107), first light transmitted through the object of interest (101) or second light emitted by a contrast agent inside the object of interest (101), resulting in detection data; and
performing, by a reconstruction unit (108), a fluoresce reconstruction on the basis of the detection data and a spectral model, resulting in reconstruction data comprising a distribution of the contrast agent inside the object of interest (101).
